(19) United States
(12) Patent Application Publication
(10) Pub. No.: US 2014/0058116 A1
(43) Pub. Date: Feb. 27, 2014

(54) PROCESS FOR PREPARING INHIBITORS OF THE HEPATITIS C VIRUS

(71) Applicants: Ji Xie, Edison, NJ (US); George Wu, Basking Ridge, NJ (US); Joseph Lamberson, Jersey City, NJ (US); Marc Poirier, Stewartsville, NJ (US); Frank Chen, Plainsboro, NJ (US)

(72) Inventors: Ji Xie, Edison, NJ (US); George Wu, Basking Ridge, NJ (US); Joseph Lamberson, Jersey City, NJ (US); Marc Poirier, Stewartsville, NJ (US); Frank Chen, Plainsboro, NJ (US)

(21) Appl. No.: 14/114,251

(22) PCT Filed: May 2, 2012
(86) PCT No.: PCT/US12/36112

§ 371 Date: Oct. 28, 2013

Related U.S. Application Data
(60) Provisional application No. 61/482,592, filed on May 4, 2011.

(51) Int. Cl. C07D 209/52

(52) U.S. Cl. CPC C07D 209/52
USPC 548/515

(57) ABSTRACT

The present invention relates to synthetic processes useful in the preparation of compounds of Formula (I) that are useful as inhibitors of the hepatitis C virus (HCV) NS3 protease and have application in the treatment of conditions caused by HCV. In particular, the present invention relates to novel oxidation processes useful for preparing compounds of Formula (I) and related compounds, including pharmaceutically acceptable salts, hydrates and solvates thereof, and including stereoisomers thereof.

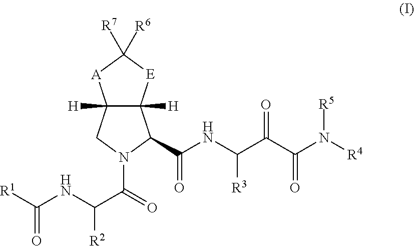

### FIELD OF THE INVENTION

The present invention relates to synthetic processes useful in the preparation of compounds that are useful as inhibitors of the hepatitis C virus (HCV) NS3 protease and have application in the treatment of conditions caused by HCV. In particular, the present invention relates to novel oxidation processes useful for preparing compounds of Formula I:

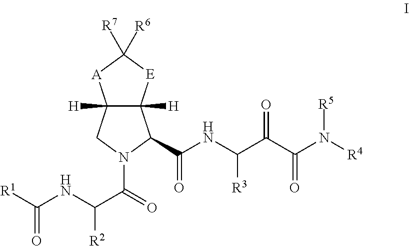

and related compounds, including pharmaceutically acceptable salts, hydrates and solvates thereof, and including stereoisomers thereof.

### BACKGROUND OF THE INVENTION

Hepatitis C virus (HCV) infection is a major health problem that leads to chronic liver disease, such as cirrhosis and hepatocellular carcinoma, in a substantial number of infected individuals. Current treatments for HCV infection include immunotherapy with recombinant interferon-α alone or in combination with the nucleoside analog ribavirin.

Several virally-encoded enzymes are putative targets for therapeutic intervention, including a metalloprotease (NS2-3), a serine protease (NS3, amino acid residues 1-180), a helicase (NS3, full length), an NS3 protease cofactor (NS4A), a membrane protein (NS4B), a zinc metalloprotein (NS5A) and an RNA-dependent RNA polymerase (NS5B). The NS3 protease is located in the N-terminal domain of the NS3 protein, and is considered a prime drug target because it is responsible for an intramolecular cleavage at the NS3/4A site and for downstream intermolecular processing at the NS4A/4B, NS4B/5A and NS5A/5B junctions.

U.S. Pat. No. 7,012,066 describes compounds that are useful as HCV NS3 inhibitors and useful in the treatment of HCV and conditions caused by HCV infection. U.S. Pat. Nos. 7,728,165, 7,723,531, 7,595,419, 7,569,705, 7,528,263, 7,326,795, 7,309,717, and 6,992,220; U.S. Patent Application Publications No. US2011/0034705, US2010/0256393, US2010/0145069, US2010/0145013, US2010/0113821, US2009/0326244 US2008/0254128, and US2008/0193518; and International Patent Application Publication WO2009/073380 describe processes for preparing such compounds. However, there is a continuing need for improved chemical processes for preparing compounds that are potent inhibitors of intermolecular cleavage at the NS3/4A site. This disclosure addresses this need.

### SUMMARY OF THE INVENTION

The present invention relates to chemical processes useful in the synthesis of compounds of Formula I and related compounds, including salts, hydrates and solvates thereof, and including stereoisomers thereof, that are useful as inhibitors of the hepatitis C virus NS3 protease.

The chemical processes of the present invention afford advantages over previously known procedures and include an efficient route to compounds of Formula I. In particular, the processes of the present invention afford a halogen-free oxidation process for preparing compounds of Formula I.

More particularly, the present invention relates to processes for preparing a compound of Formula I,

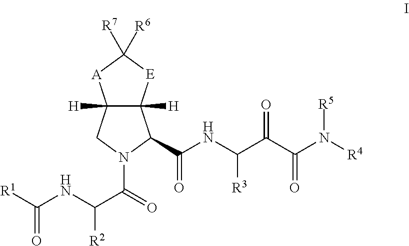

wherein:

A and E are independently selected from the group consisting of a direct bond and C₁-C₆alkylene;

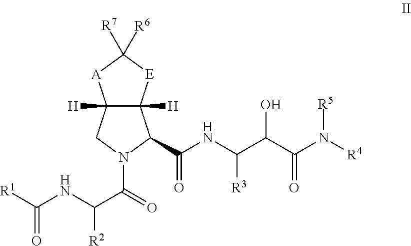

R¹ is —NH(C₁-C₈alkyl),

R² is C₁-C₈alkyl;

R³ is independently selected from the group consisting of C₁-C₈alkyl, C₁-C₈alkyl(C₃-C₈cycloalkyl) and substituted C₁-C₈alkyl(C₃-C₈cycloalkyl); or

R⁴ and R⁵ are each independently selected from the group consisting of H, C₁-C₈allcyl, C₃-C₈cycloalkyl, C₁-C₈alkyl(C₃-C₈cycloalkyl) and substituted C₁-C₈alkyl(C₃-C₈cycloalkyl),

or R⁴ and R⁵ may be taken together to form a C₃-C₈cycloalkyl;

R⁶ and R⁷ are independently H or C₁-C₈alkyl;

the process comprising:

reacting a compound of Formula II:

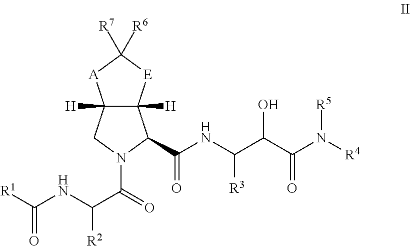

wherein A, E, R¹, R², R³, R⁴, R⁵, R⁶ and R⁷ are as defined above, with an oxidizing agent selected from the group consisting of KMnO₄, NaMnO₄, K₂FeO₄, V₂O₅, RuO₂, NaNO₂, CrO₃, K₂CrO₄, K₂Cr₂O₇, H₅PV₂Mo₁₀O₄, peroxides and PhI(OAc)₂, in the presence of at least one catalyst to yield a compound of Formula I. In embodiments, the compounds of Formula I and Formula II may be present as amorphous compounds, or as pharmaceutically acceptable salts, hydrates, solvates or stereoisomers thereof.

### DETAILED DESCRIPTION OF THE INVENTION

A first embodiment of the invention is directed to processes in which R¹ is selected from —NHCH₃, —NHCH₂CH₃, —NHCH₂CH₂CH₃, —NHCH(CH₃)₂, —NHCH₂CH₂CH₂CH₃, —NHCH(CH₃)CH₂CH₃, —NHCH₂CH(CH₃)₂, —NHC(CH₃)₃, —NHCH₂CH₂CH₂CH₂CH₃, and —NHCH₂CH₂CH₂CH₂CH₂CH₃. In different aspects of this embodiment, R¹ is —NHC(CH₃)₃, R¹ is

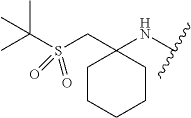 [SMILES: *NC1(CS(=O)(=O)C(C)(C)C)CCCCC1]

### or R¹ is

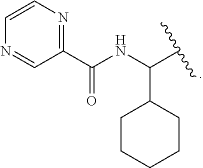 [SMILES: *C(NC(=O)c1cnccn1)C1CCCCC1]

In all aspects of this embodiment, all other groups are as provided in the general formula above.

A second embodiment of the invention is directed to processes in which R² is selected from —CH₃, —CH₂CH₃, —CH₂CH₂CH₃, —CH(CH₃)₂, —CH₂CH₂CH₂CH₃, —CH(CH₃)CH₂CH₃, —CH₂CH(CH₃)₂, —C(CH₃)₃, —CH₂CH₂CH₂CH₂CH₃, and —CH₂CH₂CH₂CH₂CH₂CH₃. In a particular aspect of this embodiment, R² is —C(CH₃)₃. In all aspects of this embodiment, all other groups are as provided in the general formula above and/or in the first embodiment.

A third embodiment of the invention is directed to processes in which R³ is selected from the group consisting of —C₁-C₈alkyl and —(CH₂)₁₋₈(cyclo(C₃-C₈)alkyl). In aspects of this embodiment, R³ is —CH₂CH₂CH₂CH₃ or

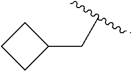 [SMILES: *CC1CCC1]

In a particular aspect of this embodiment, R³ is

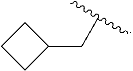 [SMILES: *CC1CCC1]

In all aspects of this embodiment, all other groups are as provided in the general formula above and/or in the first or second embodiments.

A fourth embodiment of the invention is directed to processes in which R⁴ is selected from the group consisting of H, C₁-C₈alkyl, C₃-C₈cycloalkyl, C₁-C₈alkyl(C₃-C₈ cycloalkyl) and substituted C₁-C₈alkyl(C₃-C₈cycloalkyl). In particular aspects of this embodiment, R⁴ is H or R⁴ is cyclopropyl. In all aspects of this embodiment, all other groups are as provided in the general formula above and/or in the first through third embodiments.

A fifth embodiment of the invention is directed to processes in which R⁵ is selected from the group consisting of H, C₁-C₈alkyl, C₃-C₈cycloalkyl, C₁-C₈alkyl(C₃-C₈ cycloalkyl) and substituted C₁-C₈alkyl(C₃-C₈cycloalkyl). In particular aspects of this embodiment, R⁵ is H or R⁵ is cyclopropyl. In all aspects of this embodiment, all other groups are as provided in the general formula above and/or in the first through fourth embodiments.

In a sixth embodiment, R⁴ and R⁵ are taken together to form a C₃-C₈cycloalkyl. In particular aspects of this embodiment, R⁴ and R⁵ are taken together to form a C₄-C₅cycloalkyl. In all aspects of this embodiment, all other groups are as provided in the general formula above and/or in the first through third embodiments.

A seventh embodiment of the invention is directed to processes in which R⁶ is selected from the group consisting of H or C₁-C₄alkyl. In particular aspects of this embodiment, R⁶ is H or R⁶ is methyl. In all aspects of this embodiment, all other groups are as provided in the general formula above and/or in the first through sixth embodiments.

An eighth embodiment of the invention is directed to processes in which R⁷ is selected from the group consisting of H or C₁-C₄alkyl. In particular aspects of this embodiment, R⁷ is H or R⁷ is methyl. In all aspects of this embodiment, all other groups are as provided in the general formula above and/or in the first through seventh embodiments.

A ninth embodiment of the invention is directed to processes in which A and E are independently selected from the group consisting of a bond and —CH₂—. In particular aspects of this embodiment, A and E are each independently a bond. In additional aspects of this embodiment, A and E are each independently —CH₂—. In all aspects of this embodiment, all other groups are as provided in the general formula above and/or in the first through eighth embodiments.

A tenth embodiment of the invention is directed to processes in which the catalyst is selected from the group consisting of 2,2,6,6-tetramethyl-1 -piperidinyloxy free radical (TEMPO), 4-methoxy-TEMPO, 4-amino-TEMPO, 2-azaadamantane N-oxyl (AZADO), 1-Me-AZADO and combinations of one to five catalysts chosen therefrom. In this embodiment, the catalyst may be any single catalyst selected from the group, or any two, three, four or five catalysts selected from the group set forth above. In a particular aspect of this embodiment, the catalyst is TEMPO. In aspects of this embodiment, the catalyst is present in a stoichiometric amount, with respect to the compound of Formula II. In particular aspects of this embodiment, the at least one catalyst is present in an amount ranging from about 0.1 to about 2.0 equivalents, per equivalent of the compound of Formula II. In particular aspects of this embodiment, the at least one catalyst is present in an amount ranging from about 0.6 to about 1.3 equivalents, per equivalent of the compound of Formula II. In all aspects of this embodiment, all other groups are as provided in the general formula above and/or in the first through ninth embodiments.

An eleventh embodiment of the invention is directed to processes in which the oxidizing agent is selected from the group consisting of KMnO₄, NaMnO₄, CrO₃, K₂CrO₄, K₂Cr₂O₇, and H₅PV₂Mo₁₀O₄. In particular aspects of this embodiment, the oxidizing agent is selected from the group consisting of KMnO₄, NaMnO₄, H₅PV₂Mo₁₀O₄ and K₂Cr₂O₇. In additional aspects of this embodiment, the oxidizing agent is present in an amount ranging from about 0.5 to about 1.2 equivalents, per equivalent of the compound of Formula II, and in specific aspects of this embodiment, the oxidizing agent is present in an amount ranging from about 0.6 to about 1.0 equivalents, per equivalent of the compound of Formula II. In all aspects of this embodiment, all other groups are as provided in the general formula above and/or in the first through tenth embodiments.

A twelfth embodiment of the invention is directed to processes in which the reacting is conducted in the presence of an acid. In particular aspects of this embodiment, the acid is selected from the group consisting of HCl, KHSO₄, KH₂PO₄, ClCH₂COOH, Cl₂CHCOOH, CH₃COOH and HOCH₂COOH. In additional aspects of this embodiment, the acid is provided as a 1N to 4N solution. In particular instances of these aspects of this embodiment, the acid is provided as a 2N to 4N solution. In still further aspects of this embodiment, the acid is present in an amount ranging from about 1.0 to about 20 equivalents, per equivalent of the compound of Formula II, and in specific aspects of this embodiment, the acid is present in an amount ranging from about 3.0 to about 10 equivalents, per equivalent of the compound of Formula II. In all aspects of this embodiment, all other groups are as provided in the general formula above and/or in the first through eleventh embodiments.

A thirteenth embodiment of the invention is directed to processes in which the reacting takes place at a temperature in a range of from about 0° C. to about 40° C., in particular aspects of this embodiment, in a range of from about 3° C. to about 30° C., and in still further aspects of this embodiment, in a range of from about 5° C. to about 25° C. In all aspects of this embodiment, all other groups are as provided in the general formula above and/or in the first through twelfth embodiments. A fourteenth embodiment of the invention is directed to processes in which the compound of Formula I is a compound of Formula Ia:

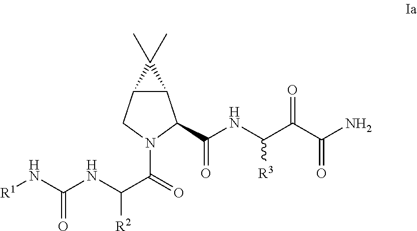

and the compound of Formula II is a compound of Formula IIa:

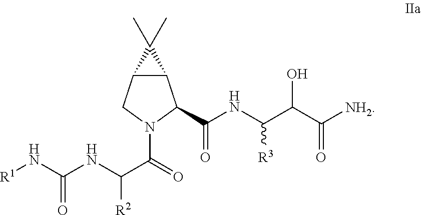

In all aspects of this embodiment, all other groups and conditions are as provided in the general formula above and/or in the first through thirteenth embodiments.

A fifteenth embodiment of the invention is directed to processes in which the compound of Formula I is a compound of Formula Ib:

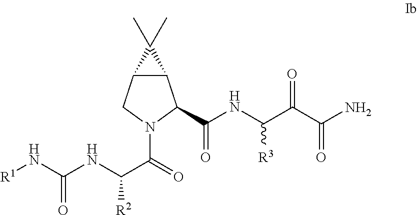

and the compound of Formula II is a compound of Formula IIb:

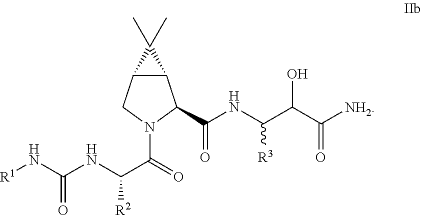

In all aspects of this embodiment, all other groups and conditions are as provided in the general formula above and/or in the first through thirteenth embodiments.

A sixteenth embodiment of the invention is directed to processes in which the compound of Formula I is a compound of Formula Ic:

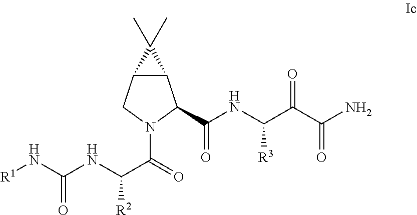

and the compound of Formula II is a compound of Formula IIc:

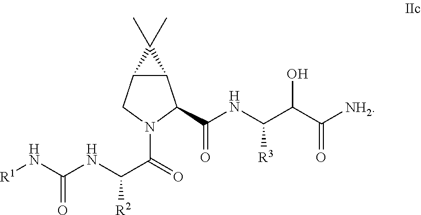

In all aspects of this embodiment, all other groups and conditions are as provided in the general formula above and/or in the first through thirteenth embodiments.

A seventeenth embodiment of the invention is directed to processes in which the compound of Formula I is a compound of Formula Id:

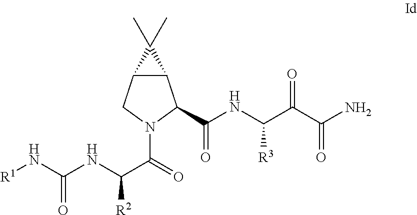

and the compound of Formula II is a compound of Formula IId:

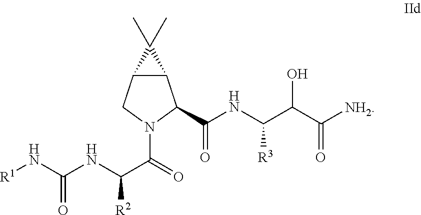

In all aspects of this embodiment, all other groups and conditions are as provided in the general formula above and/or in the first through thirteenth embodiments.

An eighteenth embodiment of the invention is directed to processes in which the compound of Formula I is a compound of Formula Ie:

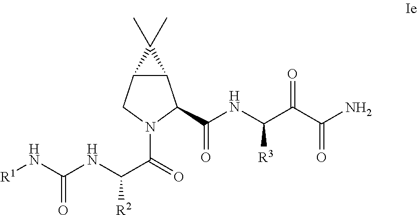

and the compound of Formula II is a compound of Formula IIe:

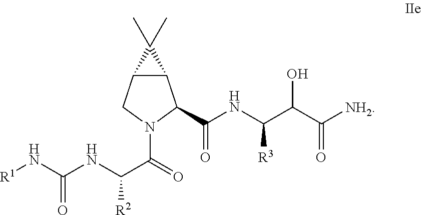

In all aspects of this embodiment, all other groups and conditions are as provided in the general formula above and/or in the first through thirteenth embodiments.

A nineteenth embodiment of the invention is directed to processes in which the compound of Formula I is a compound of Formula If:

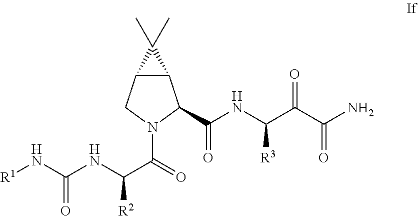

and the compound of Formula II is a compound of Formula IIf:

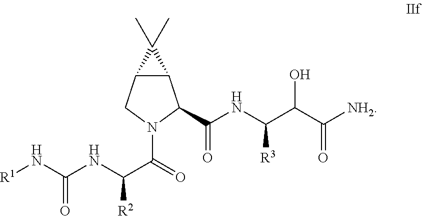

In all aspects of this embodiment, all other groups and conditions are as provided in the general formula above and/or in the first through thirteenth embodiments.

A twentieth embodiment of the invention is directed to processes in which the compound of Formula I is a compound of Formula Ig:

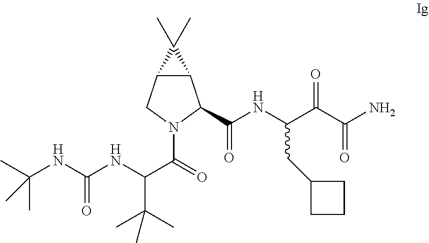 [SMILES: CC(C)(C)NC(=O)NC(C(=O)N1C[C@H]2[C@@H]([C@H]1C(=O)NC(CC1CCC1)C(=O)C(N)=O)C2(C)C)C(C)(C)C]

and the compound of Formula II is a compound of Formula IIg:

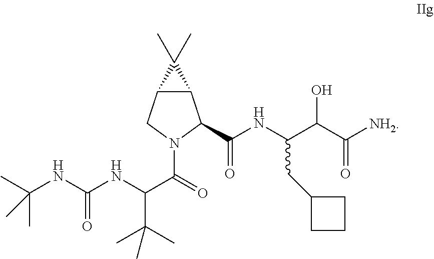 [SMILES: CC(C)(C)NC(=O)NC(C(=O)N1C[C@H]2[C@@H]([C@H]1C(=O)NC(CC1CCC1)C(O)C(N)=O)C2(C)C)C(C)(C)C]

In all aspects of this embodiment, all conditions are as provided in the general formula above and/or in the first through thirteenth embodiments.

A twenty-first embodiment of the invention is directed to processes in which the compound of Formula I is a compound of Formula Ih:

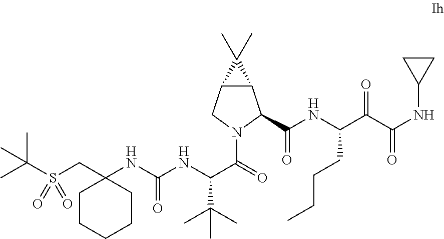 [SMILES: CCCC[C@H](NC(=O)[C@@H]1[C@@H]2[C@H](CN1C(=O)[C@@H](NC(=O)NC1(CS(=O)(=O)C(C)(C)C)CCCCC1)C(C)(C)C)C2(C)C)C(=O)C(=O)NC1CC1]

and the compound of Formula II is a compound of Formula IIh:

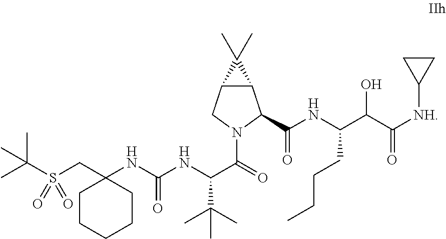 [SMILES: CCCC[C@H](NC(=O)[C@@H]1[C@@H]2[C@H](CN1C(=O)[C@@H](NC(=O)NC1(CS(=O)(=O)C(C)(C)C)CCCCC1)C(C)(C)C)C2(C)C)C(O)C(=O)NC1CC1]

In all aspects of this embodiment, all conditions are as provided in the general formula above and/or in the first through thirteenth embodiments.

A twenty-second embodiment of the invention is directed to processes in which the compound of Formula I is a compound of Formula Ii:

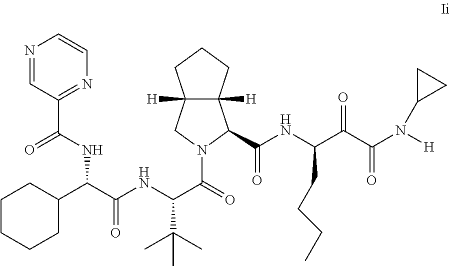 [SMILES: CCCC[C@@H](NC(=O)[C@@H]1[C@H]2CCC[C@H]2CN1C(=O)[C@@H](NC(=O)[C@@H](NC(=O)c1cnccn1)C1CCCCC1)C(C)(C)C)C(=O)C(=O)NC1CC1]

and the compound of Formula II is a compound of Formula IIi:

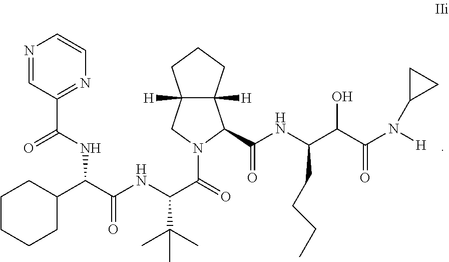 [SMILES: CCCC[C@@H](NC(=O)[C@@H]1[C@H]2CCC[C@H]2CN1C(=O)[C@@H](NC(=O)[C@@H](NC(=O)c1cnccn1)C1CCCCC1)C(C)(C)C)C(O)C(=O)NC1CC1]

In all aspects of this embodiment, all conditions are as provided in the general formula above and/or in the first through thirteenth embodiments.

A twenty-third embodiment of the invention is directed to a compound of Formula I or a pharmaceutically acceptable salt thereof, wherein the compound is prepared by the process according to any one of the general process above and/or any one of the first through twenty-second embodiments. In all aspects of this embodiment, all groups are as provided in the general process above and/or in any of the first through twenty-second embodiments above.

In a twenty-fourth embodiment of the invention, a compound of the invention is prepared by process according to any one of the general process above and/or any one of the first through twenty-second embodiments and is selected from the exemplary species depicted in Examples 2 through 4 shown below.

In the embodiments of processes for preparing the compounds and salts provided above, it is to be understood that each embodiment may be combined with one or more other embodiments, to the extent that such a combination provides a stable compound or salt and is consistent with the description of the embodiments. It is further to be understood that the embodiments of compositions and methods provided are understood to include all embodiments of the compounds and/or salts, including such embodiments as result from combinations of embodiments. Further, each of the embodiments described above, for the compounds of Formula I and Formula II, variables A, E, R¹, R², R³, R⁴, R⁵, R⁶ and R⁷ and reagents, including the oxidizing agents and catalysts are selected independently from each other.

The present invention also includes a compound of the present invention for use (i) in, (ii) as a medicament for, or (iii) in the preparation of a medicament for: (a) inhibiting HCV NS3 activity, or (b) treating HCV infection and/or reducing the likelihood or severity of symptoms of HCV infection, or (c) use in medicine. In these uses, the compounds of the present invention can optionally be employed in combination with one or more second therapeutic agents selected from HCV antiviral agents, anti-infective agents, and immunomodulators.

Additional embodiments of the invention include the pharmaceutical compositions, combinations and methods set forth above and the uses set forth in the preceding paragraph, wherein the compound of the present invention employed therein is a compound of one of the embodiments, aspects, classes, sub-classes, or features of the compounds described above. In all of these embodiments, the compound may optionally be used in the form of a pharmaceutically acceptable salt or hydrate as appropriate.

As used above, and throughout the specification, the following terms, unless otherwise indicated, shall be understood to have the following meanings:

As used herein, the term “alkyl” refers to any linear or branched chain alkyl group having a number of carbon atoms in the specified range. Thus, for example, “C₁₋₆alkyl” (or “C₁-C₆alkyl”) refers to all of the hexyl alkyl and pentyl alkyl isomers as well as n-, iso-, sec- and t-butyl, n- and isopropyl, ethyl and methyl. Alkyl groups may be substituted as indicated, by substituents that may be the same or different, each substituent being independently selected from the group consisting of halo, alkyl, aryl, cycloalkyl, cyano, hydroxy, alkoxy, alkylthio, amino, —NH(alkyl), —NH(cycloalkyl), —N(alkyl)₂, carboxy and —C(O)O-alkyl. Non-limiting examples of suitable alkyl groups include methyl, ethyl, n-propyl, isopropyl, n-butyl, t-butyl, n-pentyl, heptyl, nonyl, decyl, fluoromethyl, trifluoromethyl and cyclopropylmethyl.

The term “alkoxy” refers to an “alkyl-O-” group. Alkoxy groups may be substituted as indicated.

The term “cycloalkyl” refers to any cyclic ring of an alkane or alkene having a number of carbon atoms in the specified range. Thus, for example, “C₃₋₈cycloalkyl” (or “C₃-C₈cycloalkyl”) refers to cyclopropyl, cyclobutyl, cyclopentyl, cyclohexyl, cycloheptyl, and cyclooctyl. The term “cycloalkoxy” refers to a “cycloalkyl-O-” group. Cycloalkyl groups may be substituted as indicated.

The term “aryl” (or “aryl ring system”) refers to aromatic mono- and poly-carbocyclic ring systems wherein the individual carbocyclic rings in the polyring systems are fused or attached to each other via a single bond. As used herein, the term aryl includes aromatic mono- and poly-carbocyclic ring systems that include from 0 to 4 heteroatoms (non-carbon atoms) that are independently chosen from N, O and S. Suitable aryl groups include phenyl, naphthyl, biphenylenyl, pyridinyl, pyrimidinyl and pyrrolyl, as well as those discussed below. Aryl groups may be substituted as indicated. Aryl ring systems may include, where appropriate, an indication of the variable to which a particular ring atom is attached. Unless otherwise indicated, substituents to the aryl ring systems can be attached to any ring atom, provided that such attachment results in formation of a stable ring system.

“Halo” means fluoro, chloro, bromo, or iodo groups. Preferred are fluoro, chloro or bromo, and more preferred are fluoro and chloro. Similarly, “halogen” means fluorine, chlorine, bromine, or iodine. Preferred are fluorine, chlorine or bromine, and more preferred are fluorine and chlorine.

“Ring system substituent” means a substituent attached to an aromatic or non-aromatic ring system that, for example, replaces an available hydrogen on the ring system. Ring system substituents may be the same or different, each being independently selected from the group consisting of aryl, heteroaryl, aralkyl, alkylaryl, aralkenyl, heteroaralkyl, alkylheteroaryl, heteroaralkenyl, hydroxy, hydroxyalkyl, alkoxy, aryloxy, aralkoxy, acyl, aroyl, halo, nitro, cyano, carboxy, alkoxycarbonyl, aryloxycarbonyl, aralkoxycarbonyl, alkylsulfonyl, arylsulfonyl, heteroarylsulfonyl, alkylsulfinyl, arylsulfinyl, heteroarylsulfinyl, alkylthio, arylthio, heteroarylthio, aralkylthio, heteroaralkylthio, cycloalkyl, cycloalkenyl, heterocyclyl, heterocyclenyl, Y₁Y₂N—, Y₁Y₂N-alkyl-, Y₁Y₂NC(O)— and Y₁Y₂NSO₂—, wherein Y₁ and Y₂ may be the same or different and are independently selected from the group consisting of hydrogen, alkyl, aryl, and aralkyl.

“Cycloalkylalkyl” means a cycloalkyl-alkyl group in which the cycloalkyl and alkyl groups are as previously described. The cycloalkyl portion may be optionally substituted with one or more “ring system substituents.” The alkyl portion may be substituted with one or more alkyl substituents as defined above.

Unless otherwise specifically noted as only “substituted”, a particular group is not substituted. Preferably, the substituents are selected from the group which includes, but is not limited to, halo, C₁-C₂₀alkyl, —CF₃, —NH₂, —N(C₁-C₆ alkyl)₂, —NO₂, oxo, —CN, —N₃, —OH, —O(C₁-C₆alkyl), C₃-C₁₀cycloalkyl, C₂-C₆alkenyl, C₂-C₆ alkynyl, (C₀-C₆alkyl) S(O)₀₋₂—, aryl-S(O)₀₋₂—, (C₀-C₆alkyl)S(O)₀₋₂(C₀-C₆alkyl)—, (C₀-C₆ alkyl)C(O)NH—, H₂N—C(NH)—, —O(C₁-C₆alkyl)CF₃, (C₀-C₆alkyl)C(O)—, (C₀-C₆alkyl)OC(O)—, (C₀-C₆alkyl)O(C₁-C₆ alkyl)—, (C₀-C₆alkyl)C(O)₁₋₂(C₀-C₆alkyl)—, (C₀-C₆alkyl)OC(O)NH—, aryl, aralkyl, heteroaryl, heterocyclylalkyl, halo-aryl, halo-aralkyl, halo-heterocycle and halo-heterocyclylalkyl.

Unless expressly stated to the contrary, all ranges cited herein are inclusive. For example, a heteroaryl ring described as containing from “0 to 3 heteroatoms” means the ring can contain 0, 1, 2, or 3 heteroatoms. It is also to be understood that any range cited herein includes within its scope all of the sub-ranges within that range. The oxidized forms of the heteroatoms N and S are also included within the scope of the present invention. In addition, the term “or,” as used herein, denotes alternatives that may, where appropriate, be combined; that is, the term “or” includes each listed alternative separately as well as their combination.

Unless expressly stated to the contrary, substitution by a named substituent is permitted on any atom provided such substitution is chemically allowed and results in a stable compound. A “stable” compound is a compound that can be prepared and isolated and whose structure and properties remain or can be caused to remain essentially unchanged for a period of time sufficient to allow use of the compound for the purposes described herein (e.g., therapeutic or prophylactic administration to a subject).

As a result of the selection of substituents and substituent patterns, certain of the compounds of the present invention can have asymmetric centers and can occur as mixtures of stereoisomers, or as individual diastereomers, or enantiomers. All isomeric forms of these compounds, whether isolated or in mixtures, are within the scope of the present invention. The compounds prepared via the present invention may be chiral as a result of asymmetric centers, chiral axes, or chiral planes as described in: E. L. Eliel and S. H. Wilen, Stereochemistry of Carbon Compounds, John Wiley & Sons, New York, 1994, pages 1119-1190), and may occur as single optical isomers or as mixtures of any number of the possible optical isomers, including racemates, racemic mixtures, diastereomers, diastereomeric mixtures, enantiomers, and enantiomeric mixtures. In certain instances, the compounds disclosed may exist as tautomers and all tautomeric forms are intended to be encompassed by the scope of the invention, even though only one tautomeric structure is depicted. That is, for the purposes of the present invention, a reference to a compound of Formula I is a reference to the compound per se, or to any one of its tautomers per se, or to mixtures of two or more tautomers.

Racemic mixtures can be separated into their individual enantiomers by any of a number of conventional methods. These include chiral chromatography, derivatization with a chiral auxiliary followed by separation by chromatography or crystallization, and fractional crystallization of diastereomeric salts.

The compounds of the present invention may be administered in the form of pharmaceutically acceptable salts. The term “pharmaceutically acceptable salt” refers to a salt that possesses the effectiveness of the parent compound and that is not biologically or otherwise undesirable (e.g., is neither toxic nor otherwise deleterious to the recipient thereof). Suitable salts include acid addition salts that may, for example, be formed by mixing a solution of the compound of the present invention with a solution of a pharmaceutically acceptable acid such as hydrochloric acid, sulfuric acid, acetic acid, trifluoroacetic acid, or benzoic acid. Many of the compounds of the invention carry an acidic moiety, in which case suitable pharmaceutically acceptable salts thereof can include alkali metal salts (e.g., sodium or potassium salts), alkaline earth metal salts (e.g., calcium or magnesium salts), and salts formed with suitable organic ligands such as quaternary ammonium salts. Also, in the case of an acid (—COOH) or alcohol group being present, pharmaceutically acceptable esters can be employed to modify the solubility or hydrolysis characteristics of the compound.

The term “administration” and variants thereof (e.g., “administering” a compound) in reference to a compound of the invention mean providing the compound or a prodrug of the compound to the individual in need of treatment. When a compound of the invention or a prodrug thereof is provided in combination with one or more other active agents (e.g., antiviral agents useful for treating HCV infection), “administration” and its variants are each understood to include concurrent and sequential provision of the compound or salt (or hydrate) and other agents.

As used herein, the term “composition” is intended to encompass a product comprising the specified ingredients, as well as any product that results, directly or indirectly, from combining the specified ingredients.

By “pharmaceutically acceptable” is meant that the ingredients of the pharmaceutical composition must be compatible with each other and not deleterious to the recipient thereof.

The terms “subject” (alternatively referred to herein as “patient”) and “cell-based system”, as used herein, refer to an animal, preferably a mammal, most preferably a human, who has been the object of treatment, observation or experiment.

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, lithium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, formic, fumaric, gluconic, glutamic, hydrobromic, hydrochloric, isethionic, lactic, maleic, malic, mandelic, methanesulfonic, malonic, mucic, nitric, pamoic, pantothenic, phosphoric, propionic, succinic, sulfuric, tartaric, p-toluenesulfonic acid, trifluoroacetic acid, and the like. Particularly preferred are citric, fumaric, hydrobromic, hydrochloric, maleic, phosphoric, sulfuric, and tartaric acids.

The compounds afforded by the instant invention are useful intermediates in the production of HCV NS3 inhibitor compounds or are themselves HCV NS3 inhibitor compounds useful for treating conditions caused by HCV infection or which can be ameliorated by inhibition of HCV infection, and/or reduction of the likelihood or severity of symptoms of HCV infection, alone or in combination with other active agents. For example, the compounds of this invention are useful in treating infection by HCV after suspected past exposure to HCV by such means as blood transfusion, exchange of body fluids, bites, accidental needle stick, or exposure to patient blood during surgery. Treatment is effected by administration of the final product obtained from the disclosed processes to a mammal in need of such treatment. In addition, these compounds are useful as ingredients in pharmaceutical compositions alone or in combination with other active agents.

The following schemes and examples are illustrative of the processes encompassed by the present invention. As will be readily apparent to those in the field, the substituents and substitution patterns on the substrates exemplified herein may be modified without undue experimentation by the choice of readily available starting materials, reagents, and conventional procedures or variations. As used below and throughout this disclosure, “room temperature” or “RT” indicates that the reaction was performed at ambient temperature without the use of any means for cooling or heating. “Room temperature” is about 25° C.

The illustrative examples below, therefore, are not limited by the compounds listed or by any particular substituents employed for illustrative purposes. Substituent numbering as shown in the schemes does not necessarily correlate to that used in the claims and often, for clarity, a single substituent is shown attached to the compound in place of multiple substituents allowed under the definitions of Formula I defined above.

The processes of the instant invention are useful in the preparation of compounds of Formula I. The compounds of the present invention can be readily prepared according to the following reaction schemes and examples, or modifications thereof, using readily available starting materials, reagents and conventional synthesis procedures. In these reactions, it is also possible to make use of variants which are themselves known to those of ordinary skill in this art, but are not mentioned in greater detail. Furthermore, other methods for preparing compounds of the invention will be readily apparent to the person of ordinary skill in the art in light of the following reaction schemes and examples. Unless otherwise indicated, all variables are as defined above. The following reaction schemes and examples serve only to illustrate the invention and its practice.

### EXAMPLES

The following listing defines the abbreviations used herein, both above and in the Examples below.

### Abbreviations

¹H NMR Proton nuclear magnetic resonance spectrum

### Ac Acetyl or —C(O)CH₃

### AZADO 2-Azaadamantane N-oxyl

CrO₃ Chromium oxide
eq. Equivalents

### g Grams

### H₅PV₂Mo₁₀O₄ Polyoxymetalates

HCl Hydrochloric acid
HOAc Acetic acid or CH₃COOH
K₂Cr₂O₇ Potassium dichromate
K₂CrO₄ Potassium chromate
K₂FeO₄ Potassium ferrate
KBr Potassium bromide

### kg Kilogram

KMnO₄ Potassium permanganate

### L Liter

### M Molar

### Me Methyl or —CH₃

### mL Milliliters

mmols Millimoles
MTBE Methyl t-butyl ether

### N Normal

NaMnO₄ Sodium permanganate
NaNO₂ Sodium nitrite
NaOAc Sodium acetate
NaOCl Sodium hypochlorite (bleach)

### Ph Phenyl or —C₆H₅

### PhI(OAc)₂ (Diacetoxyiodo)benzene

rpm Revolutions per minute
RT Room temperature, approximately 25° C.
RuO₂ Ruthenium oxide
TEMPO 2,2,6,6-tetramethyl-1-piperidinyloxy free radical (available from Aldrich and used as received)
V₂O₅ Vanadium oxide

### Example 1

### (1R,2S,5S)-N-(4-amino-1-cyclobutyl-3-hydroxy-4-oxobutan-2-yl)-3-[N-(tert-butylcarbamoyl)-3-methylvalyl]-6,6-dimethyl-3-azabicyclo[3.1.0]hexane-2-carboxamide

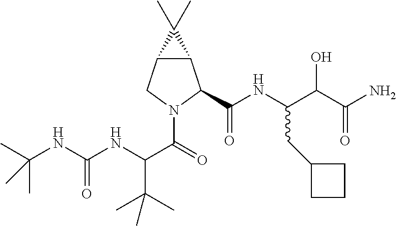 [SMILES: CC(C)(C)NC(=O)NC(C(=O)N1C[C@H]2[C@@H]([C@H]1C(=O)NC(CC1CCC1)C(O)C(N)=O)C2(C)C)C(C)(C)C]

(1R,2S,5S)-N-(4-amino-1-cyclobutyl-3-hydroxy-4-oxobutan-2-yl)-3-[N-(tert-butylcarbamoyl)-3-methylvalyl]-6,6-dimethyl-3-azabicyclo[3.1.0]hexane-2-carboxamide was prepared according to the processes disclosed in U.S. Patent Application Publication No. US2010/519485 A1, the disclosures of which are herein incorporated by reference. It will be appreciated that the processes disclosed therein can be modified without undue experimentation to prepare specifically desired starting materials.

### Example 2

### (1R,2S,5S)-N-(4-amino-1-cyclobutyl-3,4-dioxobutan-2-yl)-3-[N-(tert-butylcarbamoyl-3-methylvalyl]-6,6-dimethyl-3-azabicyclo[3.1.0]hexane-2-carboxamide

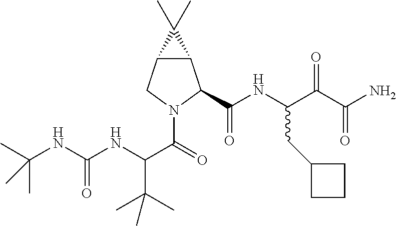 [SMILES: CC(C)(C)NC(=O)NC(C(=O)N1C[C@H]2[C@@H]([C@H]1C(=O)NC(CC1CCC1)C(=O)C(N)=O)C2(C)C)C(C)(C)C]

The compound of Example 1 (500 g), TEMPO (164.7 g), methyl tent-butyl ether (4 L) and acetic acid (472 g) were charged into a 10-L, 3-necked flask equipped with a mechanical stirrer, addition funnel and thermometer. The mixture was cooled to between 10° C. and 20° C. To the cooled mixture, pre-diluted NaMnO₄ (289 g of 40% NaMnO₄ and 1.65 L of water) was added drop-wise while maintaining the temperature between 10° C. and 20° C. The mixture was agitated while maintaining the temperature between 10° C. and 20° C. until the reaction was complete. The reaction mixture was cooled to between 0° C. and 5° C., and 500 ml of water was added. The layers were settled and separated. The organic layer was washed with 2.5 L of water and filtered to remove any solid. The organic layer was washed at 5 to 15° C. for about 4 hours with an ascorbic acid solution prepared from 500 g of sodium ascorbate, 1.655 L of water and 0.875 L of 9.9% HCl solution. After splitting the layers, the organic layer was washed with 2 L of 3.0N to 4.0N HCl solution. After separation of layers, the organic layer was washed 4 times with 2.5 L of water at between 0° C. and 10° C. The resulting organic layer was added dropwise to 15 L of n-heptane while keeping the temperature at between -10° C. and 0° C. The precipitate was filtered and dried at 35° C. to 40° C. to give the desired product. The isolated yield of desired product was 73%-90% by weight. ¹HNMR, δ0.84 (d, J=2.3 Hz, 3H), 0.90-1.02 (m, 9H), 0.99 (d, J=4.0 Hz, 3H), 1.24 (s, 9H), 1.40-1.86 (m, 7H), 1.90-2.10 (m, 3H), 2.25-2.40 (m, 1H), 3.75 (dd, J=5.3 and 10.4 Hz, 1H), 4.10 (dd, J=6.8 and 10.4 Hz, 1H), 4.4 (dd, J=3.0 and 5.3 Hz, 2H), 5.17 (dddd, J=4.6, 8.1, 8.1, and 10.4 Hz, 1H), 5.3 (br s, 2H), 6.71 (d, J=14.7 Hz, 1H), 6.90 (dd, J=2.3 and 19.0 Hz, 1H), and 7.34 (dd, J=7.1 and 20.2 Hz, 1H).

### Example 3

### (1R,2S,5S)-N-(4-amino-1-cyclobutyl-3,4-dioxobutan-2-yl)-3-[N-(tert-butylcarbamoyl)-3-methylvalyl]-6,6-dimethyl-3-azabicyclo[3.1.0]hexane-2-carboxamide

The title compound was prepared according to the procedures in Example 2, using 5.0 g of the compound of Example 1, and 0.91 g of KMnO₄, dissolved in 25 mL of water, in place of NaMnO₄. The isolated yield was about 85% by weight of a product having an identical ¹H NMR spectrum to that of the product of Example 2.

### Example 4

### (1R,2S,5S)-N-(4-amino-1-cyclobutyl-3,4-dioxobutan-2-yl)-3-[N-(tert-butylcarbamoyl)-3-methylvalyl]-6,6-dimethyl-3-azabicyclo[3.1.0]hexane-2-carboxamide

The compound of Example 1 (320 kg), TEMPO (106 kg), methyl tent-butyl ether (2560 L) and acetic acid (302 kg) were charged into a 11000 -L, glass-lined reactor that was equipped with a retreat curve impeller, temperature probes and a temperature control jacket. The mixture was cooled to a temperature between 11° C. and 22° C. To the cooled mixture, pre-diluted NaMnO₄ (181 kg of 40% NaMnO₄ and 1056 L of water) was added drop-wise over 2 to 3 hours while maintaining the temperature between 11° C. and 22° C. The mixture was agitated while maintaining the temperature between 11° C. and 22° C. until the reaction was complete. The reaction mixture was cooled to between 0° C. and 10° C., and 256 L of water was added. The layers were settled and separated. The organic layer was washed with 1600 L of water and filtered to remove any solid. The organic layer was washed at 5° C. to 15° C. for about 4 hours with an ascorbic acid solution prepared from 320 kg of sodium ascorbate, 1060 L of water and 560 kg of 9.9% HCl solution. After splitting the layers, the organic layer was washed with about 1280 L of 3.0 to 4.0N HCl solution. After separation of layers, the organic layer was washed 4 times with 1600 L of water at between 0° C. and 10° C. The resulting organic layer was precipitated by mixing it continuously with cold n-heptane (kept between −25° C. and 15° C.) by use of a tee mixer at a volumetric ratio of 1:4, while maintaining its temperature at between −10° C. and 0° C. The precipitate was distilled under vacuum by following the temperature and % batch volume distilled profile shown in Table 1 to a final volume of 10×. The batch was then filtered and dried at 35° C. to 45° C. to give the desired product. The isolated yield of desired product was 88% by weight.

TABLE 1
Distillation profile used in Example 4.

| Time (h) | Distillation Temperature (° C.) | % Batch Volume Distilled |
|---|---|---|
| 3 | 15.1 to 22.5 | 0.0 to 2.8 |
| | 17.7 to 22.5 | 2.8 to 4.1 |
| 6 | 16.3 to 22.4 | 2.8 to 7.0 |
| | 19.1 to 22.4 | 7.0 to 8.4 |
| 10 | 19.2 to 24.2 | 5.9 to 11.4 |
| | 19.2 to 24.6 | 11.4 to 14.7 |
| | 20.2 to 24.6 | 14.7 to 18.8 |

### Comparative Example 1

### (1R,2S,5S)-N-(4-amino-1-cyclobutyl-3,4-dioxobutan-2-yl)-3-[N-(tert-butylcarbamoyl)-3-methylvalyl]-6,6-dimethyl-3-azabicyclo[3.1.0]hexane-2-carboxamide by the Process of U.S. Pat. No. 7,583,263, Example 1

Into a 1 L, three-necked flask is placed KBr (10 g, 84 mmol), NaOAc (10 g, 122 mmol), the compound of Example 1 (50 g, 96 mmol), and TEMPO (15 g, 96 mmol), followed by 500 mL of MTBE. The reaction mixture is stirred at 350-400 rpm, and the temperature is maintained at a temperature of from 10° C. to 20° C. Acetic acid (50 mL, 874 mmol), and water (5 mL) are added to the reaction mixture and the two phase mixture is agitated for 15 minutes. Continuously, over a two hour period, to the reaction mixture is added 158 mL of a 0.82 M solution of NaOCl (130 mmol). When all of the NaOCl solution is added, the reaction mixture is stirred for an additional 3 hours while maintaining the temperature. Water (50 mL) is added. The layers are separated and the organic layer is washed twice with water (2×250 mL). A solution of ascorbic acid, which is prepared from 50 g of sodium ascorbate, 200 mL of water, and 50 mL of 4N HCl, is added to the organic layer and the mixture is stirred for about 1 hour. After the layers are separated, the organic layer is washed twice with water (2×250 mL). The organic layer is concentrated by distilling off solvent at low temperature (0-5° C.) until the total volume is about 350 mL. The concentrated organic layer is added dropwise over 30 minutes into a 3 L flask containing 2 L of n-heptane at about 0° C. providing a white precipitate. The white precipitate is collected by filtration, washed with n-heptane (400 mL) and dried in a vacuum oven (2 hours at 25° C., 8 hours at 35°, and 8 hours at 45° C.). The product is obtained as a white powder (typically 94-96% yield). ¹H NMR, δ0.84 (d, J=2.3 Hz, 3H), 0.90-1.02 (m, 9H), 0.99 (d, J=4.0 Hz, 3H), 1.24 (s, 9H), 1.40-1.86 (m, 7H), 1.90-2.10 (m, 3H), 2.25-2.40 (m, 1H), 3.75 (dd, J=5.3 and 10.4 Hz, 1H), 4.10 (dd, J=6.8 and 10.4 Hz, 1H), 4.4 (dd, J=3.0 and 5.3 Hz, 2H), 5.17 (dddd, J=4.6, 8.1, 8.1, and 10.4 Hz, 1H), 5.3 (br s, 2H), 6.71 (d, J=14.7 Hz, 1H), 6.90 (dd, J=2.3 and 19.0 Hz, 1H), and 7.34 (dd, J=7.1 and 20.2 Hz, 1H).

### Comparative Example 2

### (1R,2S,5S)-N-(4-amino-1-cyclobutyl-3,4-dioxobutan-2-yl)-3-[N-(tert-butykarbamoyl)-3-methylvalyl]-6,6-dimethyl-3-azabicyclo[3.1.0]hexane-2-carboxamide by the Process of U.S. Pat. No. 7,583,263, Example 2

Into a 2 L, three necked flask was charged KBr (20 g, 168 mmol), NaOAc (20 g, 243 mmol), the compound of Example 1 (100 g, 192 mmol), and TEMPO (30 g, 192 mmol), followed by 800 mL of MTBE. The reaction mixture was stirred at 350-400 rpm while the temperature of the reaction mixture was maintained at a temperature of from 10° C. to 20° C. Acetic acid (70 mL, 1223 mmol, used as received), was added and the mixture was agitated for 15 minutes additional. Continuously, over a two hour period, 315 ml of a 0.73 M solution of NaOCl (230 mmol) was added to the reaction mixture. When all of the NaOCl solution had been added, agitation was continued for an additional 3 hours. Water (100 mL) was added to the reaction mixture at the end of 3 hours. The layers were separated and the organic layer was washed once with water (500 mL). A solution of ascorbic acid, which was prepared from 100 g of sodium ascorbate, 456 mL of water, and 44 mL of 36% HCl, was added to the organic layer and the mixture was stirred for about 2 hours. The layers were separated and then a solution of 3.5N HCL was added and stirred about 30 minutes. After the layers were separated, the organic layer was washed three times with water (3×500 mL). This organic layer was then added drop-wise over 30 minutes into a 5 L flask containing 3 L of n-heptane at about −10 to about 0° C. The white precipitate was filtered, washed with n-heptane (600 mL) and dried in a vacuum oven (2 hours at 25° C., 8 hours at 35°, and 8 hours at 45° C.). The product was obtained as a white powder (93% yield). ¹H NMR, δ0.84 (d, J=2.3 Hz, 3H), 0.90-1.02 (m, 9H), 0.99 (d, J=4.0 Hz, 3H), 1.24 (s, 9H), 1.40-1.86 (m, 7H), 1.90-2.10 (m, 3H), 2.25-2.40 (m, 1H), 3.75 (dd, J=5.3 and 10.4 Hz, 1H), 4.10 (dd, J=6.8 and 10.4 Hz, 1H), 4.4 (dd, J=3.0 and 5.3 Hz, 2H), 5.17 (dddd, J=4.6, 8.1, 8.1, and 10.4 Hz, 1H), 5.3 (br s, 2H), 6.71 (d, J=14.7 Hz, 1H), 6.90 (dd, J=2.3 and 19.0 Hz, 1H), and 7.34 (dd, J=7.1 and 20.2 Hz, 1H).

Similar yields of 73-90% may be obtained for the procedures of Examples 2-4 and Comparative Examples 1-2 when conducted on a comparable scale, such as using 500 g and 100 g of the compound of Example 1 as starting material. However, the procedures of Examples 2-4 provide the desired product without the inclusion of halogenated impurities found in the products of Comparative Examples 1-2. Thus, the claimed procedures provide a process for producing compounds of Formula I having superior purity when compared to the processes of U.S. Pat. No. 7,583,263.

It will be appreciated that various of the above-discussed and other features and functions, or alternatives thereof, may be desirably combined into many other different systems or applications. Also that various presently unforeseen or unanticipated alternatives, modifications, variations or improvements therein may be subsequently made by those skilled in the art which are also intended to be encompassed by the following claims.

1. A process for preparing a compound of Formula I, 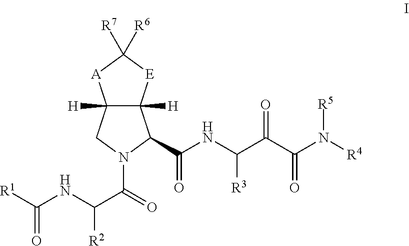
wherein:
A and E are independently a direct bond;
R¹ is —NH(C₁-C₈alkyl);
R² is C₁-C₈alkyl;
R³ is independently selected from the group consisting of C₁-C₈alkyl, C₁-C₈alkyl(C₃-C₈cycloalkyl) and substituted C₁-C₈alkyl(C₃-C₈cycloalkyl); or
R⁴ and R⁵ are each independently selected from the group consisting of H, C₁-C₈alkyl, C₃-C₈cycloalkyl, C₁-C₈alkyl(C₃-C₈cycloalkyl) and substituted C₁-C₈alkyl(C₃-C₈cycloalkyl),
or R⁴ and R⁵ may be taken together to form a C₃-C₈cycloalkyl;
R⁶ and R⁷ are independently methyl;
the process comprising:
reacting a compound of Formula II: 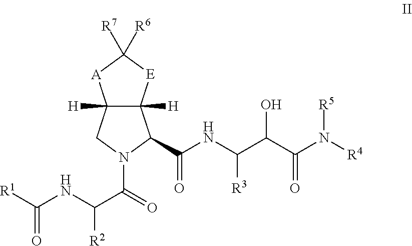
wherein A, E, R¹, R², R³, R⁴, R⁵, R⁶ and R⁷ are as defined above, with an oxidizing agent selected from the group consisting of KMnO₄, NaMnO₄, K₂FeO₄, V₂O₅, RuO₂, NaNO₂, CrO₃, K₂CrO₄, K₂Cr₂O₇, H₅PV₂Mo₁₀O₄, peroxides and PhI(OAc)₂, in the presence of at least one catalyst to yield a compound of Formula I.

2. The process according to claim 1, wherein R¹ is selected from —NHCH₃, —NHCH₂CH₃, —NHCH₂CH₂CH₃, —NHCH(CH₃)₂, —NHCH₂CH₂CH₂CH₃, —NHCH(CH₃)CH₂CH₃, —NHCH₂CH(CH₃)₂, —NHC(CH₃)₃, —NHCH₂CH₂CH₂CH₂CH₃, and —NHCH₂CH₂CH₂CH₂CH₂CH₃.

3. The process according to claim 1, wherein R² is selected from —CH₃, —CH₂CH₃, —CH₂CH₂CH₃, CH(CH₃)₂, —CH₂CH₂CH₂CH₃, —CH(CH₃)CH₂CH₃, —CH₂CH(CH₃)₂, -C(CH₃)₃, —CH₂CH₂CH₂CH₂CH₃, and —CH₂CH₂CH₂CH₂CH₂CH₃.

4. The process according to claim 1, wherein R³ is selected from the group consisting of —C₁-C₈alkyl or —(CH₂)₁₋₈(cyclo(C₃-C₈)alkyl).

5. The process according to claim 1, wherein R⁴ and R⁵ are independently selected from the group consisting of H, C₁-C₈cycloalkyl, C₁-C₈alkyl(C₃-C₈cycloalkyl) and substituted C₁-C₈alkyl(C₃-C₈cycloalkyl).

6. The process according to claim 1, wherein R⁴ and R⁵ are taken together to form a C₃-C₈cycloalkyl.

7. The process according to claim 1, wherein R⁶ and R⁷ are independently selected from the group consisting of H and C₁-C₄alkyl.

8. The process according to claim 1, wherein R¹ is —NHC(CH₃)₃, R² is —C(CH₃)₃, R³ is 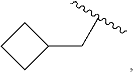 [SMILES: *CC1CCC1]
R⁴ is H, R⁵ is H, R⁶ is methyl, and R⁷ is methyl.

9. The process according to claim 1, wherein the at least one catalyst is selected from the group consisting of TEMPO, 4-methoxy-TEMPO, 4-amino-TEMPO, AZADO, 1-Me-AZADO and combinations of one to five thereof.

10. The process according to claim 9, wherein the catalyst is TEMPO.

11. The process according to claim 1, wherein the oxidizing agent is selected from the group consisting of KMnO₄, NaMnO₄, K₂Cr₂O₇, and H₅PV₂Mo₁₀O₄.

12. The process according to claim 1, wherein the oxidizing agent is present in an amount ranging from about 0.5 to about 1.2 equivalents, per equivalent of the compound of Formula II.

13. The process according to claim 1, wherein said reacting is conducted in the presence of an acid.

14. The process according to claim 13, wherein the acid is selected from the group consisting of HCl, KHSO₄, KH₂PO₄, ClCH₂COOH, Cl₂CHCOOH, CH₃COOH and HOCH₂COOH.

15. The process according to claim 13, wherein the acid is present in a concentration ranging from about 1N to about 4N.

16. The process according to claim 15, wherein the acid is present in a concentration ranging from about 2N to about 4N.

17. The process according to claim 13, wherein the acid is present in an amount ranging from about 1.0 to about 20 equivalents, per equivalent of the compound of Formula II.

18. The process according to claim 1, wherein the reacting takes place at a temperature in a range of from about 0° C. to about 40° C.

19. The process according to claim 1, wherein the compound of Formula I is a compound of Formula Ig: 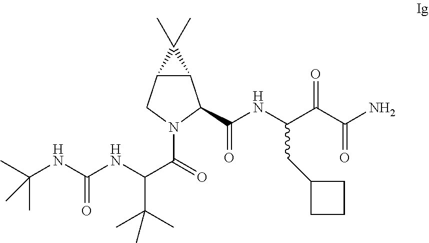 [SMILES: CC(C)(C)NC(=O)NC(C(=O)N1C[C@H]2[C@@H]([C@H]1C(=O)NC(CC1CCC1)C(=O)C(N)=O)C2(C)C)C(C)(C)C]
and the compound of Formula II is a compound of Formula IIg: 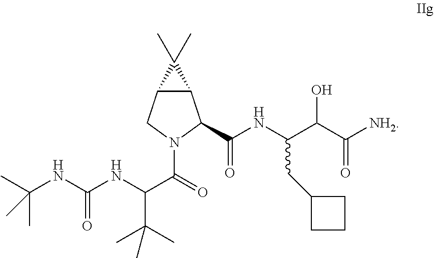 [SMILES: CC(C)(C)NC(=O)NC(C(=O)N1C[C@H]2[C@@H]([C@H]1C(=O)NC(CC1CCC1)C(O)C(N)=O)C2(C)C)C(C)(C)C]
